(19) United States
(12) Patent Application Publication
(10) Pub. No.: US 2026/0134946 A1
(43) Pub. Date: May 14, 2026

(54) NON-TRANSITORY COMPUTER-READABLE RECORDING MEDIUM, INFORMATION PROCESSING METHOD, AND INFORMATION PROCESSING DEVICE

(71) Applicants: Fujitsu Limited, Kawasaki-shi (JP); RIKEN, Wako-shi (JP)

(72) Inventors: Fuyuka YAMADA, Kawasaki (JP); Yosuke OYAMA, Kawasaki (JP); Atsushi TOKUHISA, Wako (JP); Ryo KANADA, Wako (JP); Shuntaro CHIBA, Wako (JP)

(73) Assignees: Fujitsu Limited, Kawasaki-shi (JP); RIKEN, Wako-shi (JP)

(21) Appl. No.: 19/381,246

(22) Filed: Nov. 6, 2025

(30) Foreign Application Priority Data
Nov. 8, 2024 (JP) 2024-196331

(51) Int. Cl. G16B 40/00; G06N 3/08; G16B 5/30

(52) U.S. Cl. CPC G16B 40/00; G06N 3/08; G16B 5/30

(57) ABSTRACT

A non-transitory computer-readable recording medium has stored therein an information processing program that causes a computer to execute a process including acquiring training data in which structural information of a protein is used as input data and a difference between reference energy specified based on energy corresponding to the protein and the energy is used as correct data and training a model for inferring energy of the protein based on the training data.

### CROSS-REFERENCE TO RELATED APPLICATION

This application is based upon and claims the benefit of priority of the prior Japanese Patent Application No. 2024-196331, filed on Nov. 8, 2024, the entire contents of which are incorporated herein by reference.

### FIELD

The embodiment(s) discussed herein is (are) related to a computer-readable recording medium and the like.

### BACKGROUND

In developing the drug discovery process, it is important to analyze the energy of proteins. For example, methods for calculating energy of a protein include a method using first principles calculation, a method using a classical force field, and a prediction method using a machine learning potential such as high-dimensional neural network potentials (HDNNP).

The method using the first principles calculation has features of high accuracy but high calculation cost. The method using a classical force field has features of low calculation cost but low accuracy. The prediction method using the machine learning potential can predict energy of a protein with a higher degree of freedom than that of the classical force field.

Hereinafter, description will be given on the prior art of predicting energy of a protein from structural information of the protein using a method called HDNNP. In the prior art, HDNNPs are trained using training data in which structural information of a protein is used as input data and correct energy of the protein is used as correct data.

In the prior art, energy of a protein as an evaluation target is inferred by inputting structural information of the protein as the evaluation target to a trained HDNNP.
Patent Literature 1: Japanese Laid-open Patent Publication No. 2020-101543
Patent Literature 2: International Publication Pamphlet No. WO 2022/260177
Patent Literature 3: International Publication Pamphlet No. WO 2022/260178
Patent Literature 4: U.S. Patent Application Publication No. 2022/0130496
Patent Literature 5: U.S. Patent Application Publication No. 2019/0108320

However, in the above-described prior art, there is a problem that it is difficult to improve the inference accuracy of energy of a protein.

### SUMMARY

According to an aspect of an embodiment, a non-transitory computer-readable recording medium has stored therein an information processing program that causes a computer to execute a process including acquiring training data in which structural information of a protein is used as input data and a difference between reference energy specified based on energy corresponding to the protein and the energy is used as correct data and training a model for inferring energy of the protein based on the training data

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram for explaining the structure of a protein;

FIG. 2 is a diagram illustrating energy characteristics of a protein;

FIG. 3 is a diagram illustrating an exemplary structure of an HDNNP;

FIG. 4 is a diagram for explaining the processing of training the HDNNP;

FIG. 5 is a graph (1) illustrating inference results of energy of evaluation proteins;

FIG. 6 is a graph (2) illustrating inference results of energy of evaluation proteins;

FIG. 7 is a diagram for explaining the minimum energy and a difference;

FIG. 8 is a diagram illustrating the structure of an HDNNP according to the present embodiment;

FIG. 9 is a diagram illustrating an example of training data set according to the present embodiment;

FIG. 10 is a diagram for explaining the processing of training the HDNNP according to the present embodiment;

FIG. 11 is a diagram (1) illustrating inference accuracy of the present invention compared with that of the prior art;

FIG. 12 is a diagram (2) illustrating inference accuracy of the present invention compared with that of the prior art;

FIG. 13 is a diagram illustrating a difference in characteristics of correct data between the prior art and the present invention;

FIG. 14 is a functional block diagram illustrating the configuration of an information processing device according to the present embodiment;

FIG. 15 is a flowchart illustrating a processing procedure of the information processing device according to the present embodiment; and

FIG. 16 is a diagram illustrating an example of the hardware configuration of a computer that implements functions similar to those of the information processing device according to the embodiment.

### DESCRIPTION OF EMBODIMENTS

Preferred embodiments of the present invention will be explained with reference to accompanying drawings. Note that the present invention is not limited by the embodiments.

Before describing the information processing device according to the present embodiment, proteins, the structure of HDNNPs, training of HDNNPs, and problems of the prior art will be described more specifically.

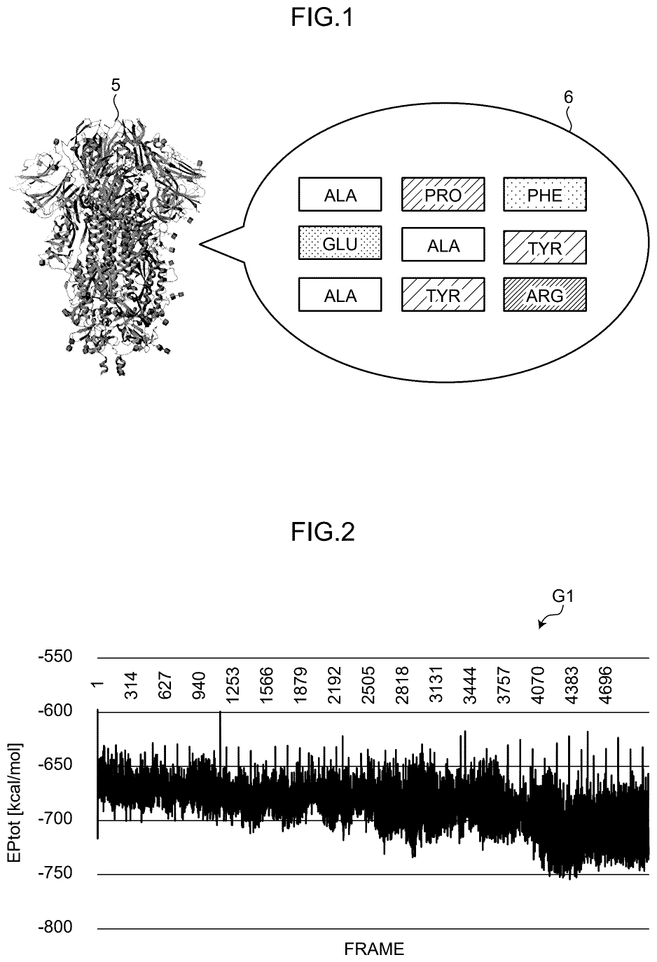

First, proteins will be explained. FIG. 1 is a diagram for explaining the structure of a protein. A protein is composed of a plurality of amino acid residues. For example, a protein 5 illustrated in FIG. 1 is composed of about 3000 amino acid residues. Examples of the constituent amino acid residues include those illustrated in a balloon 6. The balloon 6 includes three ALAs and two TYRs. The protein 5 includes one each of GLU, PRO, PHE, and ARG.

The energy characteristics of a protein will be explained. FIG. 2 is a diagram illustrating energy characteristics of a protein. The vertical axis of the graph G1 illustrated in FIG. 2 corresponds to the energy of the protein, and the horizontal axis corresponds to the frame (time). As illustrated in the graph G1, the structure of the protein changes with the lapse of time, and the energy also changes with the lapse of time. As the positions of particles included in the protein are stabilized, the energy decreases.

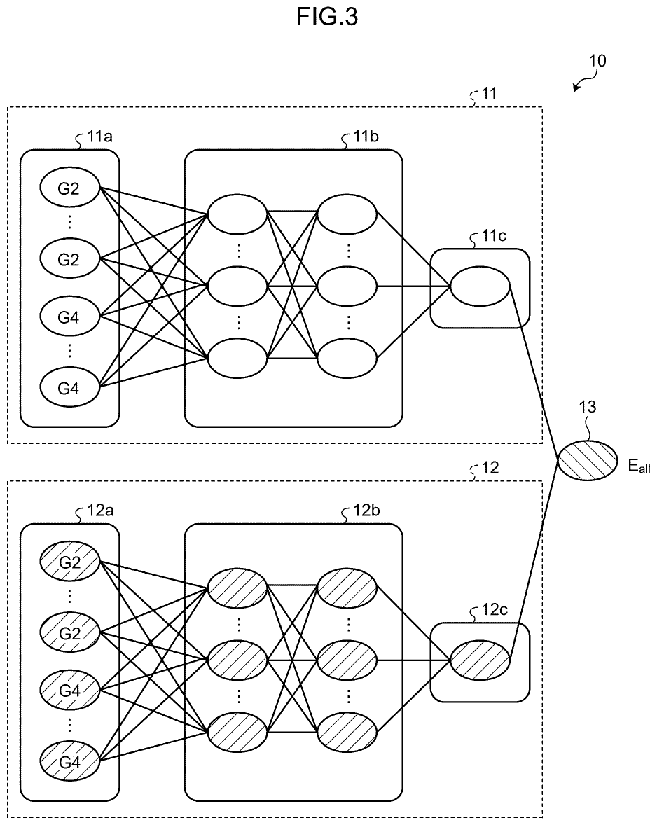

Next, the structure of an HDNNP will be described. In the HDNNP, a neural network (NN) is set for each residue type. FIG. 3 is a diagram illustrating an exemplary structure of an HDNNP. An HDNNP 10 illustrated in FIG. 3 includes an NN 11 for ALA and an NN 12 for PRO.

The NN 11 for ALA includes an input layer 11a, a hidden layer 11b, and an output layer 11c. The NN 12 for PRO includes an input layer 12a, a hidden layer 12b, and an output layer 12c. Values output from the output layers 11c and 12c are output to a summing node 13.

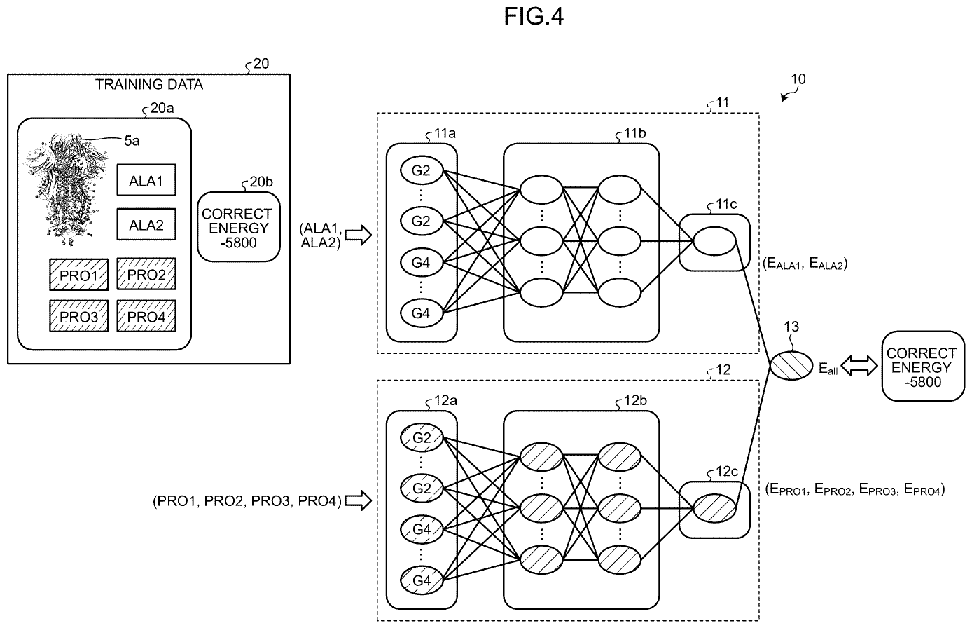

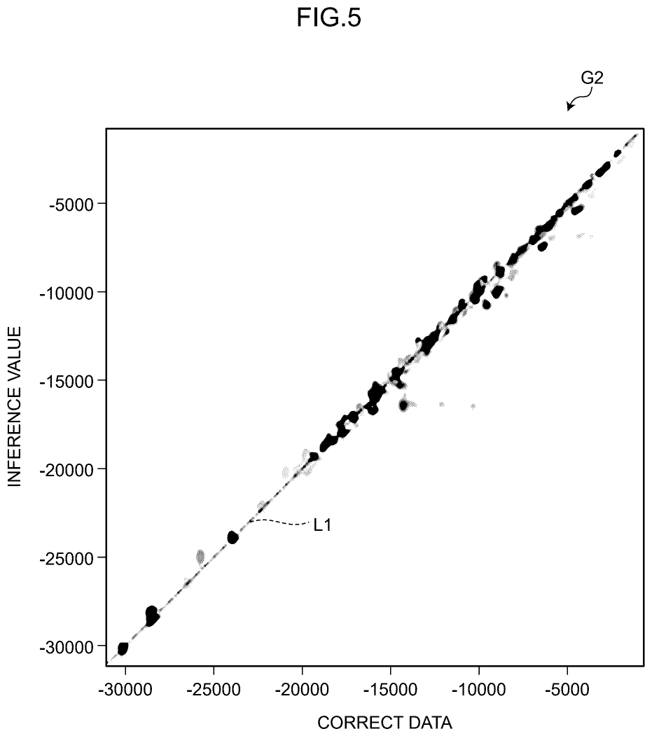

Subsequently, the prior art for training the HDNNP 10 described in FIG. 3 will be described. FIG. 4 is a diagram describing the prior art for training the HDNNP. A device for training HDNNPs is simply referred to as a “device”. The device trains the HDNNP 10 using training data 20. The training data 20 includes input data 20a and correct data 20b.

The input data 20a includes structural information of a protein 5a. For example, the protein 5a includes, as amino acid residues, ALA1, ALA2, PRO1, PRO2, PRO3, and PRO4. A correct energy of “−5800” of the protein 5a is set as the correct data 20b. Note that the protein 5a represents a state of the protein 5 at a certain time point.

The device inputs structural information of ALA1 and ALA2 (ALA1, ALA2) to the input layer 11a of the NN 11 for ALA. (ALA1, ALA2) is a sequence. Due to restriction in the description of the specification, “[” and “]” are replaced with “(“and”)”, respectively (the same applies to other sequences). The form of (ALA1, ALA2) is (2, 64). As a result, the ALA energy (E_{ALA1}, E_{ALA2}) is output from the output layer 11c of the NN 11 for ALA. (E_{ALA1}, E_{ALA2}) is a sequence and has a form of (2,1).

The device inputs structural information of PRO 1 to PRO 4 (PRO1, PRO2, PRO3, PRO4) to the input layer 12a of the NN 12 for PRO. (PRO1, PRO2, PRO3, PRO4) is a sequence and the form is (4, 64). As a result, PRO energy (E_{PRO1}, E_{PRO2}, E_{PRO3}, E_{PRO4}) is output from the output layer 12c of the NN 12 for PRO. (E_{PRO1}, E_{PRO2}, E_{PRO3}, E_{PRO4}) is a sequence and has a form of (4, 1).

The summing node 13 calculates an energy Eₐii of all residues obtained by summing (E_{ALA1}, E_{ALA2}) and (E_{PRO1}, E_{PRO2}, E_{PRO3}, E_{PRO4}). The device updates parameters of the NN 11 for ALA and the NN 12 for PRO such that an error between the energy Eau and the correct energy “−5800” becomes small. For example, the device utilizes backpropagation when updating a parameter.

The device trains the HDNNP 10 by repeatedly executing the above processing using a plurality of pieces of training data registered in the training data set.

Evaluation data is used to evaluate the trained HDNNP 10. The evaluation data includes structural information of proteins not used for the training and correct energy of such proteins. In the following description, proteins that are not used for training are referred to as “evaluation proteins”.

The device inputs the structural information of the evaluation proteins into the trained HDNNP 10. The closer the energy output from the HDNNP 10 is to the correct energy of the evaluation data, the higher the inference accuracy of the trained HDNNP 10.

Note that, as the structural information of the protein input to the HDNNP 10, a descriptor that quantifies the characteristics of the particle sequence of the protein is used. In an HDNNP, a particle arrangement around each particle is expressed by a descriptor using weighted atom-centered symmetry functions (wACSFs).

In the descriptor of the weighted atom-centered symmetry functions (wACSFs), G²₁ (radial symmetry function) and G⁴₁ (angular symmetry function) are used.

G²₁ is defined by Equation (1). G²₁ is obtained by adding up contributions corresponding to distances Rᵢⱼ between a particle i and other particles j. A term of g(Zⱼ) included in Equation (1) is a function for performing weighting by the type of particle (the type of amino acid in the present embodiment) and is defined by Equation (2). Zⱼ in Equation (2) denotes the residue type of a residue j, and Mj denotes the mass of the residue j. f_{c} included in Equation (1) denotes a cutoff function and is defined by Equation (3). The cutoff function is an attenuation function for performing calculation of the symmetry function within a range of a radius R_{C}.

$${G}_{i}^{2} = {\sum }_{i \neq 1} g ( {Z}_{j} ) {e}^{- {η ( {T}_{ij} - {R}_{s} )}^{2}} \cdot {f}_{c} ( {R}_{ij} ) \qquad ( 1 )$$

$$g ( {Z}_{j} ) = {M}_{j} \qquad ( 2 )$$

$${f}_{c} ( {R}_{ij} ) = \{ \begin{matrix}0.5 [ cos ( \frac{π {R}_{ij}}{{R}_{c}} ) + 1 ] & ( {R}_{ij} \leq {R}_{c} ) \\ 0 & ( {R}_{ij} > {R}_{c} )\end{matrix} \qquad ( 3 )$$

G⁴₁ is defined by Equation (4). G⁴₁ includes information of an angle θᵢⱼₖ formed by the particle i and two particles j and k around the particle i. Normally, by preparing a plurality of symmetric functions having different hyperparameters η, R_{S}, ζ, and λ, a reduction in the amount of information of the symmetric functions due to summation is addressed. h(Zⱼ, Zₖ) represents a function for performing weighting by the type of particle (in this example, the type of amino acid) and is defined by Equation (5). Mⱼ and Mₖ in Equation (5) denote the mass of residues j and k, respectively.

$${G}_{i}^{4} = {2}^{\text{?} - ζ} {\sum }_{j , k \neq i} h ( {Z}_{j} , {Z}_{k} ) {( 1 + λ cos {θ}_{ijk} )}^{ζ} \cdot {e}^{- η ( {( {R}_{ij} - {R}_{s} )}^{2} + {( {R}_{ik} - {R}_{s} )}^{\text{?}} + {( {R}_{jk} - {R}_{s} )}^{\text{?}} )} \cdot {f}_{c} ( {R}_{ij} ) \cdot {f}_{c} ( {R}_{ik} ) \cdot {f}_{c} ( {R}_{jk} ) \qquad ( 4 )$$

$$h ( {Z}_{j} , {Z}_{k} ) = {M}_{j} {M}_{k} \qquad ( 5 )$$

$${\text{?} \text{indicates text missing or illegible when filed}}^{}$$

Next, problems of the prior art will be described. For example, a case will be described in which the HDNNP 10 was trained on the basis of structural information of about 3500 types of proteins and the trained HDNNP 10 was evaluated using about 180 evaluation proteins.

FIG. 5 is a diagram (1) illustrating inference results of energy of the evaluation proteins. The vertical axis of the graph G2 illustrated in FIG. 5 corresponds to the inference value when the structural information of the evaluation proteins is input to the HDNNP 10. The horizontal axis of the graph G2 is the correct data (correct energy) of the evaluation proteins.

One plot of the graph G2 indicates a relationship between an inference value of an evaluation protein having certain structural information and correct data. There are a plurality of pieces of structural information for the same evaluation protein. For example, in FIG. 5, 50 types of structural information are set for each of about 180 types of proteins to perform inference, which gives a number of plots of about 9000. The larger the number of plots close to a line L1 indicating x=y, the higher the inference accuracy of the HDNNP 10.

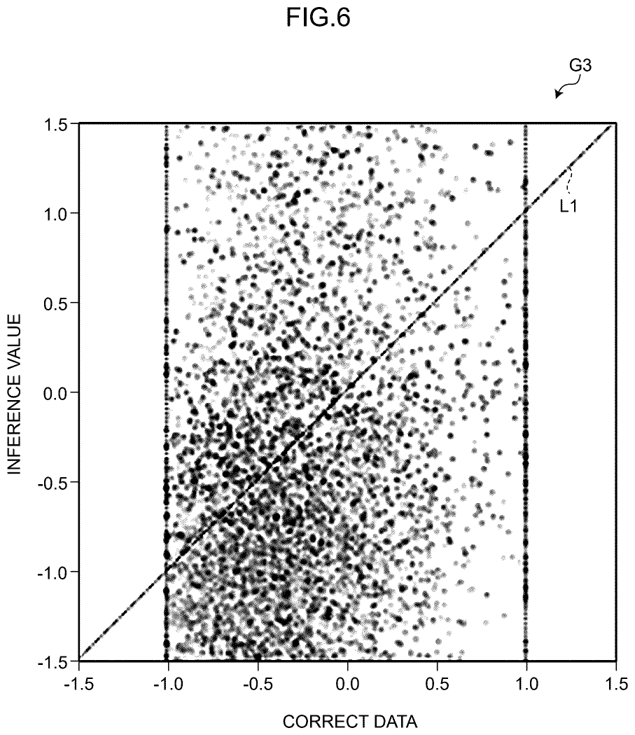

FIG. 6 is a diagram (2) illustrating inference results of energy of the evaluation proteins. The description regarding the vertical axis and the horizontal axis of the graph G3 illustrated in FIG. 6 is similar to the description regarding the vertical axis and the horizontal axis of the graph G2 in FIG. 5. In the graph G3, the relationship between the inference value and the correct data is normalized to −1 to 1 and plotted for each protein (each set of protein and structural information).

For example, as described in FIG. 2, even in the case of the same protein, the structure changes and the energy also changes with the lapse of time. However, as illustrated in FIG. 6, it can be seen that the energy for each piece of structural information of the evaluation proteins are accurately estimated in the prior art. That is, it is not possible to accurately estimate the energy change due to temporal changes.

Next, an information processing device according to the present embodiment will be described. In the following description, the information processing device according to the present embodiment will be referred to as an “information processing device 100”. As described above, in the prior art, correct energy of a protein is used as it is as correct data of training data used at the time of training. On the other hand, the information processing device 100 uses “the minimum energy of protein” and “a difference from the minimum energy” as the correct data of training data used at the time of training.

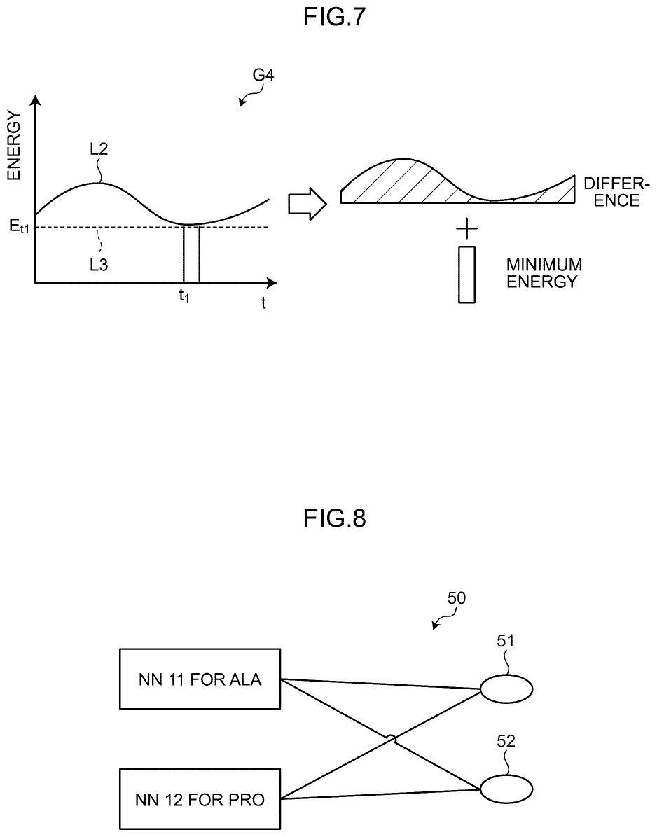

FIG. 7 is a diagram for explaining the minimum energy and the difference. A graph G4 in FIG. 7 illustrates the energy change of a certain protein with a lapse of time. The vertical axis of the graph G4 corresponds to the energy, and the horizontal axis corresponds to the time (t). A line L2 indicates the energy change of a certain protein with a lapse of time.

In the example illustrated in FIG. 7, the minimum energy of the protein is the energy Eₜ, at time t1. The difference is the value of an area surrounded by the line L3 passing through the minimum energy (energy Eₜ₁) and the line L2.

Next, the structure of the HDNNP used by the information processing device 100 will be described. FIG. 8 is a diagram illustrating the structure of the HDNNP according to the present embodiment. As illustrated in FIG. 8, an NN for each residue type is set in the HDNNP according to the present embodiment. An HDNNP 50 illustrated in FIG. 8 includes an NN 11 for ALA, an NN 12 for PRO, and summing nodes 51 and 52. In the present embodiment, only the NNs for ALA and PRO are used for convenience; however, actually, NNs for other amino acids such as LYN or GLY may also be included.

The description of the NN 11 for ALA and the NN 12 for PRO is similar to the description of the NN 11 for ALA and the NN 12 for PRO described in FIG. 3. Note that an output layer 11c of the NN 11 for ALA may include a node that outputs the minimum energy E1_{ALA} and a node that outputs a difference E2_{ALA}. Similarly, an output layer 12c of the NN 12 for PRO may include a node that outputs the minimum energy E1_{PRO} and a node that outputs a difference E2_{PRO}.

The output layer 11c of the NN 11 for ALA outputs the minimum energy E1_{ALA} to the summing node 51. The output layer 12c of the NN 12 for PRO outputs the minimum energy E1_{PRO} to the summing node 51.

The output layer 11c of the NN 11 for ALA outputs the difference E2_{ALA} to the summing node 52. The output layer 12c of the NN 12 for PRO outputs the difference E2_{PRO} to the summing node 52.

Next, an example of processing in which the information processing device 100 trains the HDNNP 50 described with reference to FIG. 8 will be described. First, an example of training data set used by the information processing device 100 will be described. Here, a case where training is performed with three samples for proteins A and B each including two ALAs and one PRO will be described as an example. Here, as an example, a case where three samples are learned for the proteins A and B will be described; however, the proteins A and B may contain amino acids other than ALA and PRO, and the proteins A and B may be composed of amino acids other than ALA and PRO. In addition, for example, the proteins A and B may contain different amino acids such that the protein A is composed of ALA and PRO, and the protein B is composed of LYS and GLY.

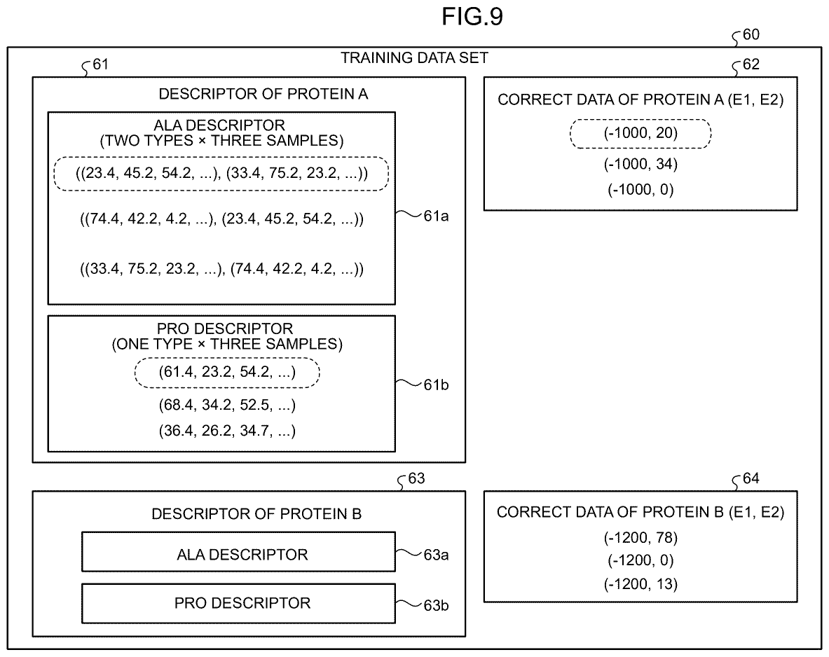

FIG. 9 is a diagram illustrating an example of training data set according to the present embodiment. In the example illustrated in FIG. 9, the training data set 60 includes a descriptor 61 of the protein A, correct data 62 of the protein A, a descriptor 63 of the protein B, and correct data 64 of the protein B.

The descriptor 61 of the protein A includes an ALA descriptor 61a and a PRO descriptor 61b. The ALA descriptor 61a includes three sample descriptors for each of two types of ALA. The PRO descriptor 61b includes three samples of descriptors for one type of PRO.

The correct data of the protein A includes three pieces of correct data (E1 minimum energy, E2 difference).

A set of input data and correct data when the HDNNP 50 is trained using the training data set 60 in FIG. 9 is as follows. For example, “(23.4, 45.2, 54.2, . . . ), (33.4, 75.2, 23.2, . . . )” of the ALA descriptor 61a and “(61.4, 23.2, 54.2, . . . )” of the PRO descriptor 61b are input data. The correct data of the protein A corresponding to such input data is “(−1000, 20)”.

“(74.4, 42.2, 4.2, . . . ), (23.4, 45.2, 54.2, . . . )” of the ALA descriptor 61a and “(68.4, 34.2, 52.5, . . . )” of the PRO descriptor 61b are input data. The correct data of the protein A corresponding to such input data is “(−1000, 34)”.

“(33.4, 75.2, 23.2, . . . ), (74.4, 42.2, 4.2, . . . )” of the ALA descriptor 61a and “(36.4, 26.2, 34.7, . . . )” of the PRO descriptor 61b are input data. The correct data of the protein A corresponding to such input data is “(−1000, 0)”.

Description of details of an ALA descriptor 63a and a PRO descriptor 63b of the descriptor 63 of the protein B will be omitted. Input data and correct data are associated with each other similarly to the descriptor 61 of the protein A.

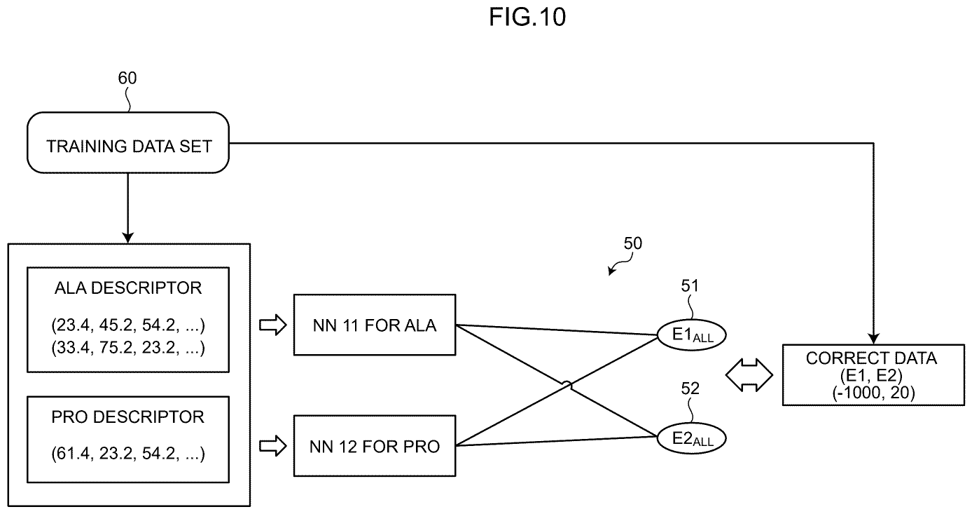

FIG. 10 is a diagram for explaining the processing of training the HDNNP according to the present embodiment. The information processing device 100 trains the HDNNP 50 using the training data set 60. The information processing device 100 acquires “(23.4, 45.2, 54.2, . . . ), (33.4, 75.2, 23.2, . . . )” of the ALA descriptor 61a from the training data set 60 and inputs the acquired data to the input layer 11a of the NN 11 for ALA, whereby the minimum energy E1_{ALA} and the difference E2_{ALA} of ALA are output. The minimum energy E1_{ALA} of ALA is output to the summing node 51. The difference E2_{ALA} of ALA is output to the summing node 52.

The information processing device 100 acquires “(61.4, 23.2, 54.2, . . . )” of the PRO descriptor 61b from the training data set 60 and inputs the acquired data to the input layer 12a of the NN 12 for PRO, whereby the minimum energy E1_{PRO} and the difference E2_{PRO} of PRO are output. The minimum energy E1_{PRO} of PRO is output to the summing node 51. The difference E2_{PRO} of PRO is output to the summing node 52.

The summing node 51 calculates E1_{ALL} obtained by summing the minimum energy of ALA, E1_{ALA}, and the minimum energy of PRO, E1_{PRO}. The summing node 52 calculates E2_{ALL} obtained by summing the difference E2_{ALA} of ALA and the difference E2_{PRO} of PRO.

The information processing device 100 updates parameters of the NN 11 for ALA and the NN 12 for PRO such that the difference value between E1_{ALL} and the correct data of “−1000” and the difference value between E2_{ALL} and the correct data of “20” become small.

The information processing device 100 also executes similar processing to the above for a set of other input data included in the descriptor 61 of the protein A of the training data set 60 and correct data included in the correct data 62 of the protein A to update the parameters of the NN 11 for ALA and the NN 12 for PRO.

Furthermore, the information processing device 100 also executes similar processing to the above for a set of other input data included in the descriptor 63 of the protein B of the training data set 60 and correct data included in the correct data 64 of the protein B to update the parameters of the NN 11 for ALA and the NN 12 for PRO.

The information processing device 100 repeatedly executes the above processing until a termination condition is satisfied. For example, the termination condition is that the number of epochs reaches a predetermined number. Alternatively, the termination condition is that the inference accuracy of the HDNNP 50 using the evaluation data is higher than or equal to a target accuracy.

For example, the evaluation data includes structural information of proteins (evaluation structural information), minimum energy of an evaluation target (evaluation minimum energy), and a difference (evaluation difference). The information processing device 100 inputs the evaluation structural information to the HDNNP 50 and estimates the minimum energy and the difference. The information processing device determines that the inference accuracy is higher than or equal to the target accuracy in a case where the difference between the estimated minimum energy and the evaluation minimum energy is less than a first threshold value and the difference between the estimated difference and the evaluation difference is less than a second threshold value.

The processing in which the information processing device 100 according to the present embodiment trains the HDNNP 50 has been described above.

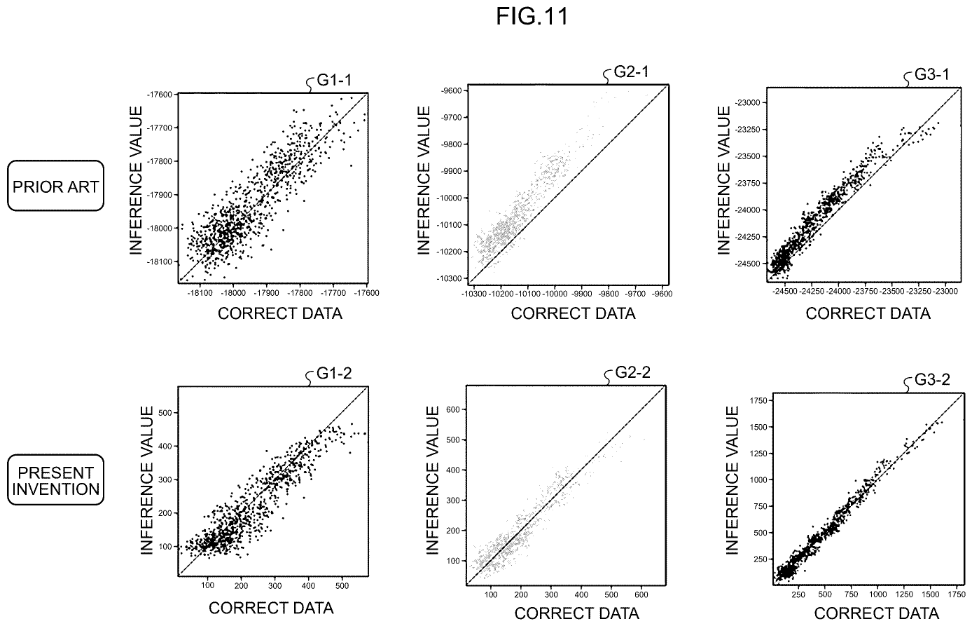

Next, a difference in inference accuracy when an inference result of the HDNNP 50 trained as in FIG. 10 is compared with an inference result of the prior art will be described. FIG. 11 is a diagram (1) illustrating inference accuracy of the present invention compared with that of the prior art. In FIG. 11, training was performed using 80 types of proteins, and (so-called interpolation) results obtained by evaluating inference accuracy using the proteins used for the training are illustrated.

An HDNNP trained by the prior art is referred to as the HDNNP 10, and an HDNNP trained by the information processing device 100 is referred to as the HDNNP 50. As described above, in the prior art, correct energy of a protein is used as it is as correct data. On the other hand, in the information processing device 100, “minimum energy of the protein” and “difference from the minimum energy” are used as correct data.

A graph G1-1 illustrates the relationship between inference values when structural information of a protein C was input to the HDNNP 10 and correct data. A graph G1-2 illustrates the relationship between inference values when the structural information of the protein C was input to the HDNNP 50 and correct data.

A graph G2-1 illustrates the relationship between inference values when structural information of a protein D was input to the HDNNP 10 and correct data. A graph G2-2 illustrates the relationship between inference values when the structural information of the protein D was input to the HDNNP 50 and correct data.

A graph G3-1 illustrates the relationship between inference values when structural information of a protein E was input to the HDNNP 10 and correct data. A graph G3-2 illustrates the relationship between inference values when the structural information of the protein E was input to the HDNNP 50 and correct data.

For example, the proteins C, D, and E are included in the training data set. Comparing the graphs G1-1 and G1-2, the graphs G2-1 and G2-2, and the graphs G3-1 and G3-2, it can be seen that the inference accuracy of the present invention has a better evaluation result than the inference accuracy of the prior art.

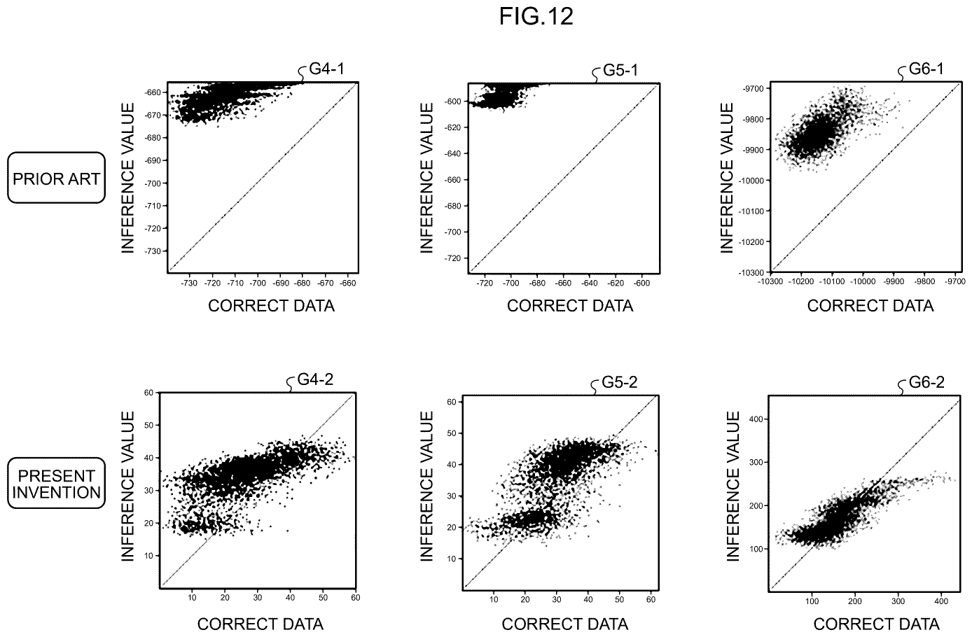

FIG. 12 is a diagram (2) illustrating inference accuracy of the present invention compared with that of the prior art. In FIG. 12, training was performed using 80 types of proteins, and (so-called extrapolation) results obtained by evaluating inference accuracy using evaluation proteins not used for the training are illustrated.

A graph G4-1 illustrates the relationship between inference values when structural information of an evaluation protein X was input to the HDNNP 10 and correct data. A graph G4-2 illustrates the relationship between inference values when the structural information of the evaluation protein X was input to the HDNNP 50 and correct data.

A graph G5-1 illustrates the relationship between inference values when structural information of an evaluation protein Y was input to the HDNNP 10 and correct data. A graph G5-2 illustrates the relationship between inference values when the structural information of the evaluation protein Y was input to the HDNNP 50 and correct data.

A graph G6-1 illustrates the relationship between inference values when structural information of an evaluation protein Z was input to the HDNNP 10 and correct data. A graph G6-2 illustrates the relationship between inference values when the structural information of the evaluation protein Z was input to the HDNNP 50 and correct data.

For example, the evaluation proteins X, Y, and Z are not included in the training data set. Comparing the graphs G4-1 and G4-2, the graphs G5-1 and G5-2, and the graphs G6-1 and G6-2, it can be seen that the inference accuracy of the present invention has a better evaluation result than the inference accuracy of the prior art even in the case of evaluating the extrapolation.

Next, a difference between characteristics of correct data according to the prior art and characteristics of correct data used in the present invention will be examined.

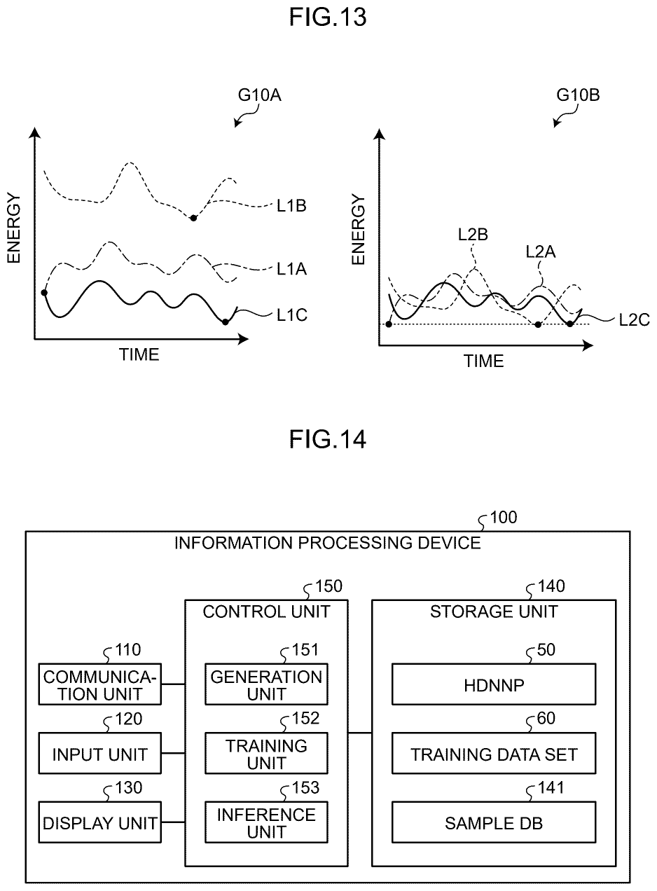

FIG. 13 is a diagram illustrating a difference in characteristics of correct data between the prior art and the present invention. A graph G10A in FIG. 13 illustrates the energy change in correct data of each type of protein with a lapse of time of the prior art. The vertical axis of G10A corresponds to energy, and the horizontal axis corresponds to time. For example, a line L1A represents correct data of the protein A of the prior art. A line L1B represents correct data of the protein B of the prior art. A line L1C represents correct data of the protein C of the prior art. Note that, in this example, the horizontal axis of G10A represents time; however, the axis may relate to other parameters.

As illustrated in the graph G10A, the energy of each protein fluctuates on different scales. Therefore, in the case of predicting energy of the same type of protein, there is no problem with performing training using such correct data; however, in the case of predicting energy of different proteins (evaluation proteins), this causes a decrease in the inference accuracy.

On the other hand, a graph G10B of FIG. 13 illustrates the energy change with a lapse of time of the correct data (in this example, the difference) of each protein of the present invention. The vertical axis of G10B corresponds to energy, and the horizontal axis corresponds to time. For example, a line L2A represents correct data of the protein A of the present invention. A line L2B represents correct data of the protein B of the present invention. A line L2C represents correct data of the protein C of the present invention.

As illustrated in the graph G10B, the energy of each protein fluctuates on a similar scale. Therefore, even in a case where energy of different types of proteins (evaluation proteins) is predicted, the inference accuracy can be improved.

Next, a configuration example of the information processing device 100 described above will be described. FIG. 14 is a functional block diagram illustrating the configuration of the information processing device according to the present embodiment. As illustrated in FIG. 14, the information processing device 100 includes a communication unit 110, an input unit 120, a display unit 130, a storage unit 140, and a control unit 150.

The communication unit 110 executes data communication with an external device and the like via a network. Furthermore, the communication unit 110 may receive the training data set 60 and the like from an external device.

The input unit 120 inputs various types of information to the control unit 150.

The display unit 130 displays information output from the control unit 150.

The storage unit 140 includes the HDNNP 50, the training data set 60, and a sample DB 141. The storage unit 140 is a memory or the like.

The HDNNP 50 is a machine learning model in which structural information of a protein is used as input and the minimum energy of the protein and a difference are used as output. Other description of the HDNNP 50 is similar to that of the HDNNP 50 described with reference to FIG. 8 and others.

The training data set 60 includes a plurality of pieces of training data for training the HDNNP 50. The training data that is input is structural information of proteins. Correct data of the training data is correct data of the minimum energy of the protein and correct data of the difference. Other description regarding the training data set 60 is similar to that regarding the training data set 60 described in FIGS. 7, 9, and the like.

The sample DB 141 has structural information of a plurality of proteins as samples. The data structure of the structural information of the proteins may be a descriptor.

The control unit 150 includes a generation unit 151, a training unit 152, and an inference unit 153. The control unit 150 is a central processing unit (CPU), a graphics processing unit (GPU), or the like.

The generation unit 151 generates the training data set 60 on the basis of the sample DB 141. For example, the generation unit 151 acquires structural information of the protein A from the sample DB 141 and calculates a change in the energy of the protein A with a lapse of time on the basis of the structural information. For example, the generation unit 151 executes a molecular dynamics (MD) simulation and calculates a change in the energy in a certain period of time.

The generation unit 151 specifies the minimum energy and the difference on the basis of the calculated energy change in the certain period of time. The generation unit 151 registers, in the training data set 60, input data as the structural information of the protein A and correct data corresponding to the minimum energy and the difference of the protein A.

The generation unit 151 generates the training data set 60 by repeatedly executing the above processing also for other proteins registered in the sample DB 141.

In this example, the case where the generation unit 151 generates the training data set 60 from the sample DB 141 has been described; however, the training data set 60 may be prepared in advance.

The training unit 152 trains the HDNNP 50 on the basis of back propagation using the training data set 60. For example, the training unit 152 acquires training data from the training data set 60, inputs input data included in the training data to the HDNNP 50, and updates parameters of the HDNNP 50 in such a manner that output from the HDNNP 50 approaches the correct data. Other description regarding the training unit 152 is similar to the processing described in FIG. 10.

The inference unit 153 infers energy of a protein using the HDNNP 50 trained by the training unit 152. For example, the inference unit 153 inputs the structural information of the protein to be inferred to the HDNNP 50 and infers the minimum energy of the protein and the difference. The inference unit 153 infers the energy of the protein by summing the inferred minimum energy and the difference. The inference unit 153 outputs and displays the inference result on the display unit 130.

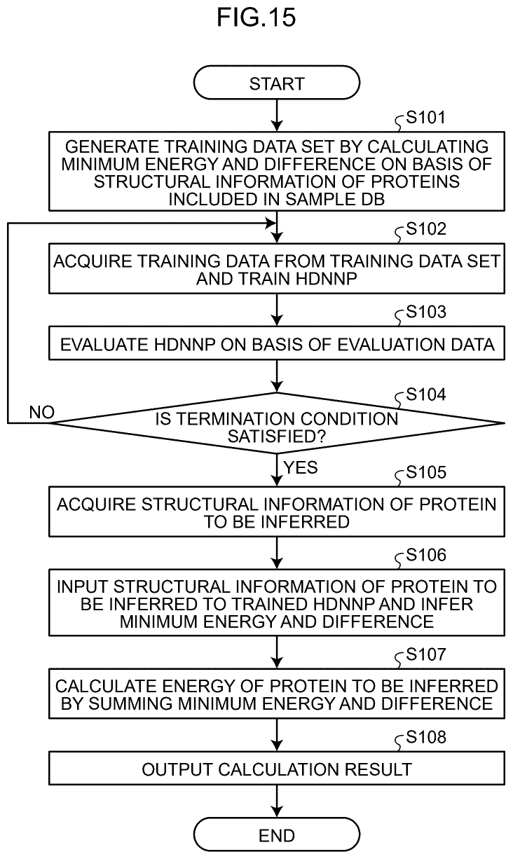

Next, an exemplary processing procedure of the information processing device 100 according to the present embodiment will be described. FIG. 15 is a flowchart illustrating a processing procedure of the information processing device according to the present embodiment. As illustrated in FIG. 15, the generation unit 151 of the information processing device 100 calculates the minimum energy and the difference on the basis of the structural information of proteins included in the sample DB 141 and generates the training data set 60 (step S101).

The training unit 152 of the information processing device 100 acquires training data from the training data set 60 and trains the HDNNP 50 (step S102). The training unit 152 evaluates the HDNNP 50 on the basis of the evaluation data (step S103). Note that the training data set 60 in FIG. 14 is divided into training data and evaluation data.

The training unit 152 determines whether or not the termination condition is satisfied (step S104). If the termination condition is not satisfied (step S104, No), the training unit 152 proceeds to step S102. On the other hand, if the termination condition is satisfied (step S104, Yes), the training unit 152 proceeds to step S105.

The inference unit 153 of the information processing device 100 acquires structural information of a protein to be inferred (step S105). The inference unit 153 inputs the structural information of the protein to be inferred to the trained HDNNP 50 and infers the minimum energy and the difference (step S106).

The inference unit 153 calculates the energy of the protein to be inferred by summing the minimum energy and the difference (step S107). The inference unit 153 outputs the calculation result (step S108).

Next, effects of the information processing device 100 according to the present embodiment will be described. The information processing device 100 trains the HDNNP 50 on the basis of training data in which structural information of proteins is used as input data and the minimum energy and the difference of the proteins are used as correct data. This makes it possible to generate the HDNNP 50 having higher protein estimation accuracy than the HDNNP 10 of the prior art.

The information processing device 100 infers the minimum energy and the difference of the protein to be inferred by inputting the structural information of the protein to be inferred to the trained HDNNP 50, and infers the energy by summing the minimum energy and the difference. With such processing, the inference accuracy can be improved as described with reference to FIGS. 11 and 12.

Incidentally, the processing content of the information processing device 100 described above is an example, and the information processing device 100 may execute other processing. For example, the information processing device 100 uses the “minimum energy of the proteins” and the “difference from the minimum energy” as the correct data used as the training data; however, it is not limited thereto. The information processing device 100 may use the “maximum energy of proteins” and “a difference from the maximum energy” or an “average energy of proteins” and “a difference from the average energy” as the correct data. The minimum energy, the maximum energy, and the average energy of the proteins correspond to “reference energy”. In the following description, the minimum energy, the maximum energy, and the average energy of the proteins are referred to as reference energy. Note that the reference energy is not limited to the above, and median energy or mode energy may be used.

Furthermore, the information processing device 100 uses a set of “reference energy of proteins” and “difference from the reference energy” as the correct data to be used as training data; however, it is not limited thereto. The information processing device 100 may use only the “difference from the reference energy” as the correct data to be used as the training data.

As described above, in a case where only the “difference from the reference energy” is used as the correct data to be used as the training data, the inference value output from the trained HDNNP 50 is only the difference from the reference energy.

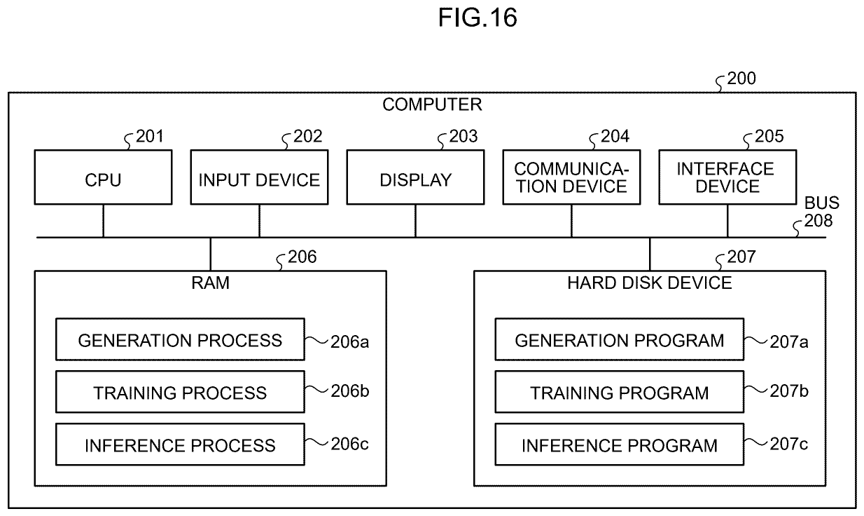

Next, an example of the hardware configuration of a computer that implements functions similar to those of the information processing device 100 described above will be described. FIG. 16 is a diagram illustrating an example of the hardware configuration of a computer that implements functions similar to those of the information processing device of the embodiment.

As illustrated in FIG. 16, a computer 200 includes a CPU 201 that executes various types of arithmetic processing, an input device 202 that receives input of data from a user, and a display 203. The computer 200 further includes a communication device 204 that exchanges data with an external device and the like via a wired or wireless network and an interface device 205. In addition, the computer 200 includes a RAM 206 that temporarily stores various types of information and a hard disk device 207. Each of the devices 201 to 207 is connected to a bus 208.

The hard disk device 207 includes a generation program 207a, a training program 207b, and an inference program 207c. The CPU 201 reads the programs 207a to 207c and develops the programs in the RAM 206.

The generation program 207a functions as a generation process 206a. The training program 207b functions as a training process 206b. The inference program 207c functions as an inference process 206c.

The processing of the generation process 206a corresponds to the processing by the generation unit 151. The processing of the training process 206b corresponds to the processing by the training unit 152. The processing of the inference process 206c corresponds to the processing by the inference unit 153.

Note that the programs 207a to 207c do not necessarily need to be stored in the hard disk device 207 from the beginning. For example, the programs are stored in a “portable physical medium” such as a flexible disk (FD), a CD-ROM, a DVD, a magneto-optical disk, or an IC card inserted into the computer 200. The computer 200 may read and execute the programs 207a to 207c.

The inference accuracy of energy of a protein can be improved.

All examples and conditional language recited herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A non-transitory computer-readable recording medium having stored therein an information processing program that causes a computer to execute a process comprising:
acquiring training data in which structural information of a protein is used as input data and a difference between reference energy specified based on energy corresponding to the protein and the energy is used as correct data; and
training a model for inferring energy of the protein based on the training data.

2. The non-transitory computer-readable recording medium according to claim 1, wherein the correct data of the training data further includes the reference energy, and the process further includes training the model based on the training data in which the reference energy is further included in the correct data.

3. The non-transitory computer-readable recording medium according to claim 2, wherein the reference energy is any one of minimum energy, maximum energy, average energy, median energy, or mode energy specified based on the energy corresponding to the protein.

4. The non-transitory computer-readable recording medium according to claim 1, wherein the process further includes inferring energy of a protein to be evaluated by acquiring structural information of the protein to be evaluated and inputting structural information of the protein to be evaluated to the model trained by the training processing.

5. The non-transitory computer-readable recording medium according to claim 1, wherein the process further includes training a high-dimensional neural network potential (HDNNP) as the model.

6. The non-transitory computer-readable recording medium according to claim 1, wherein the energy corresponding to the protein is time-series energy of the protein.

7. An information processing method comprising:
acquiring training data in which structural information of a protein is used as input data and a difference between reference energy specified based on energy corresponding to the protein and the energy is used as correct data; and
training a model for inferring energy of the protein based on the training data, by using a processor.

8. The information processing method according to claim 7, wherein the correct data of the training data further includes the reference energy, and the information processing method is further includes training the model based on the training data in which the reference energy is further included in the correct data.

9. The information processing method according to claim 8, wherein the reference energy is any one of minimum energy, maximum energy, average energy, median energy, or mode energy specified based on the energy corresponding to the protein.

10. The information processing method according to claim 7, further including inferring energy of a protein to be evaluated by acquiring structural information of the protein to be evaluated and inputting structural information of the protein to be evaluated to the model trained by the training processing.

11. The information processing method according to claim 7, further including training a high-dimensional neural network potential (HDNNP) as the model.

12. The information processing method according to claim 7, wherein the energy corresponding to the protein is time-series energy of the protein.

13. An information processing device comprising:
a memory; and
a processor coupled to the memory and configured to:
acquire training data in which structural information of a protein is used as input data and a difference between reference energy specified based on energy corresponding to the protein and the energy is used as correct data; and
train a model for inferring energy of the protein based on the training data.

14. The information processing device according to claim 13, wherein the correct data of the training data further includes the reference energy, and the processor is further configured to train the model based on the training data in which the reference energy is further included in the correct data.

15. The information processing device according to claim 14, wherein the reference energy is any one type of energy out of minimum energy, maximum energy, or average energy specified based on time-series energy of the protein.

16. The information processing device according to claim 13, wherein the processor is further configured to infer energy of a protein to be evaluated by acquiring structural information of the protein to be evaluated and inputting structural information of the protein to be evaluated to the model trained by the training processing.

17. The information processing device according to claim 13, wherein the processor is further configured to training a high-dimensional neural network potential (HDNNP) as the model.

18. The information processing device according to claim 13, wherein the energy corresponding to the protein is time-series energy of the protein.
